(19) United States
(12) Patent Application Publication
(10) Pub. No.: US 2022/0022421 A1
(43) Pub. Date: Jan. 27, 2022

(54) Freezable Pet Harness

(71) Applicant: Pearl Maremont, Pacific Palisades, CA (US)

(72) Inventor: Pearl Maremont, Pacific Palisades, CA (US)

(21) Appl. No.: 17/382,230

(22) Filed: Jul. 21, 2021

Related U.S. Application Data
(60) Provisional application No. 63/054,711, filed on Jul. 21, 2020.

(51) Int. Cl. A01K 13/00; A01K 27/00; A61D 99/00

(52) U.S. Cl. CPC A01K 13/006; A61D 99/00; A01K 27/002

(57) ABSTRACT

A freezable pet harness is an apparatus that cools down the pet's body while also comforting the pet during hot days. The apparatus may include a back-bracing pad, a neck-bracing strap, a girth-bracing band, and at least one primary gel pack. The back-bracing pad corresponds to the portion of the apparatus covering the pet's back. The neck-bracing strap secures the apparatus to the pet's neck to prevent the apparatus from getting loose. The girth-bracing band keeps the back-bracing pad positioned against the pet's back while also securing the apparatus to the pet's body. The at least one primary gel pack enables the apparatus to remain cool enough to maintain the pet's body cool when the apparatus is being worn by the pet. The at least one primary gel pack also weights down the back-bracing pad against the pet's body so that the apparatus can also serve as a gel-weighted blanket.

The current application claims a priority to the U.S. provisional patent application Ser. No. 63/054,711 filed on Jul. 21, 2020.

### FIELD OF THE INVENTION

The present invention relates generally to pet accessories and cooling devices. More specifically, the present invention provides a one-piece, dual-purpose, freezable, gel-weighted cooling, and calming dog harness capable of keeping the pet cool and calm at the same time, thus preventing heat-related issues.

### BACKGROUND OF THE INVENTION

Pets, especially dogs, love to be outdoors. For dogs in general, exercise is a crucial part to maintain a good health, resulting in the necessity for most owners to walk their dogs daily. Thus, a wide variety of pet restraints have been developed over the years which enable the dog owner to comfortably restrain their dogs. Such devices keep the dogs close to the user to prevent the dogs from running off, getting into dangerous situations, or getting lost. These harnesses and restraints also provide sufficient support to prevent the animal or pet from being hurt during use, such as when the pet pulls on the leash. However, many of these leashes and dog/pet restraints are limited in their usefulness. While often efficient in meeting their primary objective, these restraints are often thick which can make the pet uncomfortable in hot days which in turn can cause anxiety in the dogs. Dogs are especially susceptible to overheating, so furthering a dog's discomfort with a body covering restraint in hot weather is not ideal. What is needed is a pet restraint that can simultaneously keep a pet under control of the owner and cool the restrained pet. Further, a pet restraint that prevents the pet from getting hurt while pulling on the leash, is easily removable and resizable is desirable.

An objective of the present invention is to address these issues by providing a one-piece, dual-purpose, and freezable pet harness that contains a superior gel-weighted cooling material to prevent pets from overheating and to help the pets with any anxiety caused by overheating. Another objective of the present invention is to provide a freezable pet harness that is easy to put on the pet and enables size adjustment to accommodate the size of the pet. The present invention utilizes several straps with adjustable fasteners that enable the user to put the present invention on a variety of pets with relative ease. Likewise, removal is a much easier process than fumbling with various straps and buckles in similar products. Another objective of the present invention is to provide a gel-weighted, one-piece, freezable pet harness that utilizes a gel with a low thermal conductivity. This enables the user to place the present invention into a freezer between uses, thus ensuring the present invention remains cool. The gel material remains cold without requiring a change in physical state, thus enabling the present invention to conform to the pet's back and rest comfortably in position there. Additional features and benefits are further discussed in the sections below.

### SUMMARY OF THE INVENTION

The present invention provides a freezable pet harness. The freezable pet harness is a one-piece harness containing gel material that enables the freezing of the harness to cool down the pet when the present invention is being worn by the pet. The present invention serves as a dual-purpose apparatus as the freezable pet harness also serves as a gel-weighted harness due to the weight of the gel material along with the other components of the present invention. The gel-weighted capacity of the present invention helps with any anxiety issues of the pet by calming the pet with comfortable, secure sensory feelings for the pet. Therefore, the freezable pet harness not only keeps the pet cool in the outdoors during hot days but also comforts the pet. The freezable pet harness is designed to fit different pets of different sizes. Further, the present invention can be provided in different ornamental designs.

### BRIEF DESCRIPTION OF THE DRAWINGS

FIG. 1 is a front right perspective view of the present invention, wherein a dog is shown wearing the freezable pet harness.

FIG. 2 is a front right perspective view of the present invention, wherein another dog is shown wearing the freezable pet harness, and wherein a leash is shown attached to the at least one leash-attachment eyelet.

FIG. 3 is a front left perspective view of the present invention, wherein another dog is shown wearing the freezable pet harness, and wherein a leash is shown attached to the at least one leash-attachment eyelet.

FIG. 4 is a top front perspective view of the present invention, wherein the freezable pet harness is shown in a flat configuration, wherein the neck-bracing strap is shown unfastened, and wherein the girth-bracing band is shown unfastened.

FIG. 5 is a bottom rear perspective view of the present invention, wherein the freezable pet harness is shown in the flat configuration, wherein the neck-bracing strap is shown unfastened, and wherein the girth-bracing band is shown unfastened.

FIG. 6 is a side view of the present invention, wherein the freezable pet harness is shown in the flat configuration, wherein the neck-bracing strap is shown unfastened, and wherein the girth-bracing band is shown unfastened.

FIG. 7 is a vertical cross-sectional view of the present invention taken in direction of line 7-7 in FIG. 6.

FIG. 8 is a schematic view showing the at least one primary gel pack positioned within the back-bracing pad.

FIG. 9 is a schematic view showing the at least one secondary gel pack positioned within the girth-bracing band.

### DETAIL DESCRIPTIONS OF THE INVENTION

All illustrations of the drawings are for the purpose of describing selected versions of the present invention and are not intended to limit the scope of the present invention.

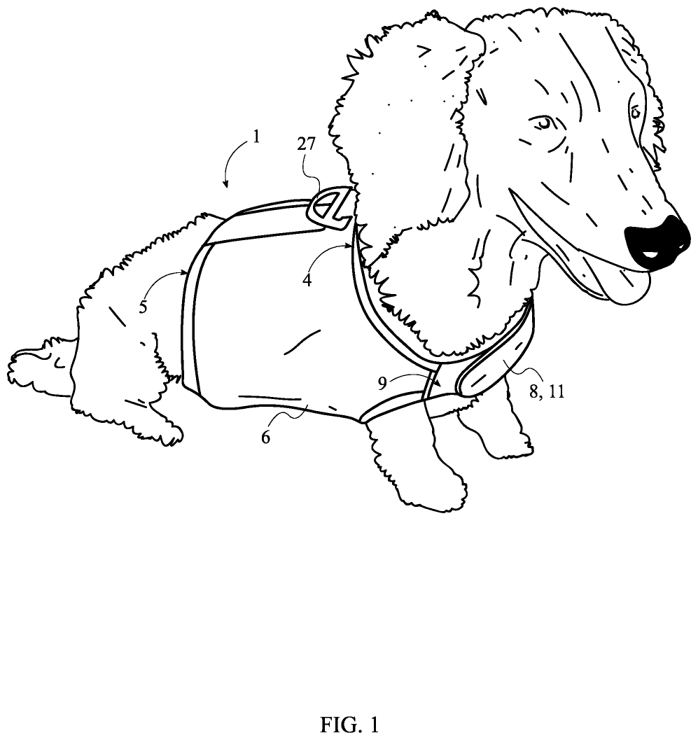

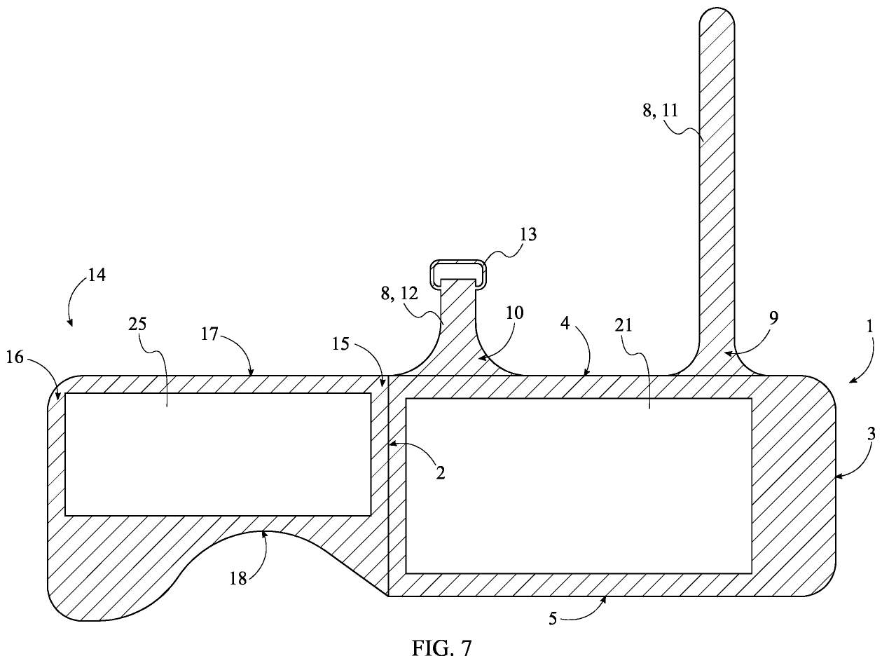

The present invention is a freezable pet harness that cools down the pet's body while also comforting the pet. As can be seen in FIGS. 1, 2, 7, and 8, the present invention may comprise a back-bracing pad 1, a neck-bracing strap 8, a girth-bracing band 14, and at least one primary gel pack 21. The back-bracing pad 1 corresponds to the portion of the present invention covering the pet's back. The neck-bracing strap 8 secures the present invention to the pet's neck to prevent the present invention from getting loose. The girth-bracing band 14 keeps the back-bracing pad 1 positioned against the pet's back while also securing the present invention to the pet's body. The at least one primary gel pack 21 enables the present invention to be frozen to maintain the pet's body cool. The at least one primary gel pack 21 also weights down the back-bracing pad 1 against the pet's body so that the present invention can serve as a gel-weighted blanket. Together, these components form a single flexible structure that comfortably wraps around the body of the pet to cool and comfort the pet.

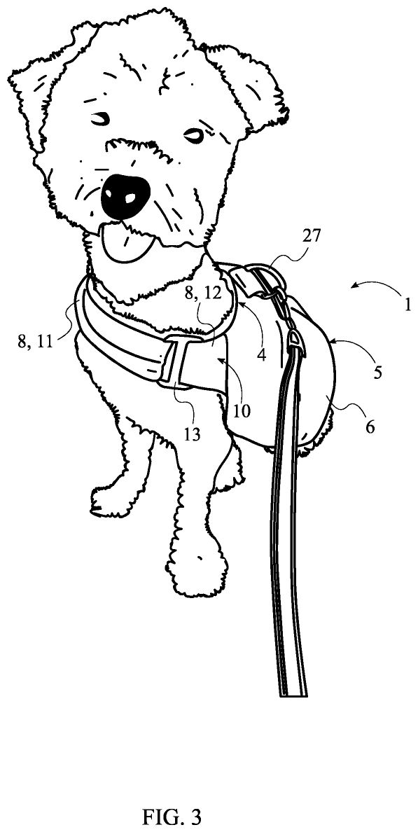

The general configuration of the aforementioned components enables easy attachment of the present invention to the body of the pet to keep the pet cool and comfortable. As can be seen in FIG. 1 through 3, 4, and 5, the back-bracing pad 1 is designed to conform to the back of the pet's body. The back-bracing pad 1 comprises a first lateral edge 2, a second lateral edge 3, a cranial edge 4, and a caudal edge 5. The first lateral edge 2 and the second lateral edge 3 preferably correspond to the shorter side edges of the back-bracing pad 1. The cranial edge 4 preferably corresponds to the edge that is positioned adjacent to the pet's neck. The caudal edge 5 preferably corresponds to the edge that is positioned adjacent to the pet's rear. Further, the neck-bracing strap 8 is an elongated strap structure that is comfortably wrapped around the pet's neck. The neck-bracing strap 8 comprises a first strap end 9 and a second strap end 10 corresponding to the terminal strap ends of the neck-bracing strap 8. Similarly, the girth-bracing band 14 is an elongated, wide band structure that fits around the pet's abdomen. Furthermore, the girth-bracing band 14 comprises a first band end 15 and a second band end 16 corresponding to the terminal ends of the girth-bracing band 14.

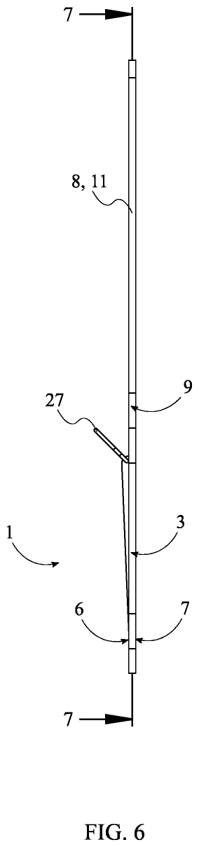

As previously mentioned, the present invention is provided as a single harness structure that can be easily operated by the pet owner. As can be seen in FIG. 1 through 3, 4, and 5, the first lateral edge 2 and the second lateral edge 3 are positioned opposite to each other across the back-bracing pad 1 due to the width of the back-bracing pad 1. Similarly, the cranial edge 4 and the caudal edge 5 are positioned opposite to each other across the back-bracing pad 1 due to the length of the back-bracing pad 1. Further, the cranial edge 4 and the caudal edge 5 are positioned in between the first lateral edge 2 and the second lateral edge 3 to form an overall elongated rectangular structure. As can be seen in FIG. 6 through 8, the at least one primary gel pack 21 is integrated into the back-bracing pad 1 to enable the present invention to cool down the pet. The first strap end 9 and the second strap end 10 are connected adjacent to the cranial edge 4 to secure the back-bracing pad 1 to the pet's neck. In addition, the first strap end 9 and the second strap end 10 are positioned offset from each other due to accommodate the pet's neck. Furthermore, the first band end 15 is connected adjacent to the first lateral edge 2. The second band end 16 is attached adjacent to the second lateral edge 3. Thus, the girth-bracing band 14 can be wrapped around the pet's abdomen to secure the back-bracing pad 1 to the pet's body.

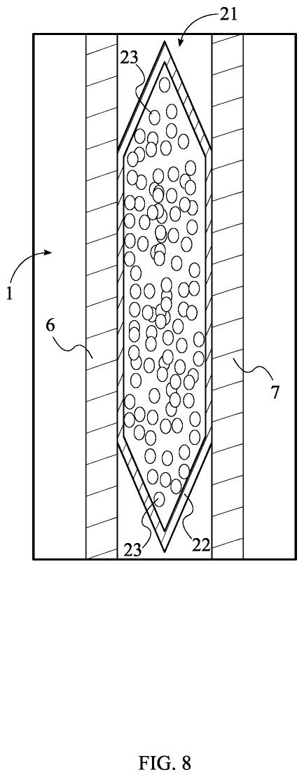

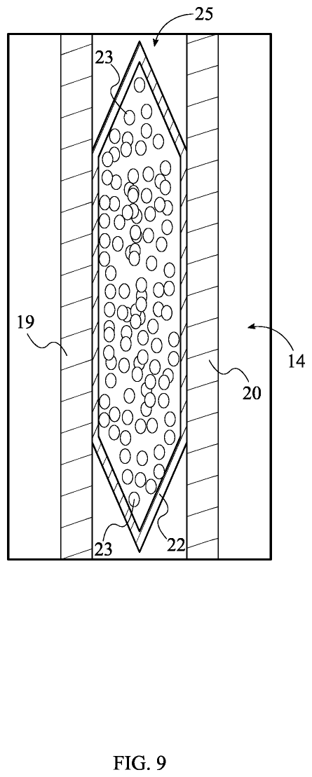

To make the present invention a freezable device, the at least one primary gel pack 21 is designed to be placed inside a refrigerator or similar cooling device so that the at least one primary gel pack 21 can be frozen. In addition, the at least one primary gel pack 21 is designed to be reusable so that the present invention can be frozen multiple times. So, the at least one primary gel pack 21 may comprise a primary hermetically-sealed pouch 22 and a primary quantity of water-based refrigerant gel 23, as can be seen in FIGS. 8 and 9. The primary hermetically-sealed pouch 22 serves to contain the primary quantity of water-based refrigerant gel 23 inside the back-bracing pad 1 so that the primary quantity of water-based refrigerant gel 23 does not leak. The primary quantity of water-based refrigerant gel 23 is designed to be frozen multiple times without the primary quantity of water-based refrigerant gel 23 changing phase. The primary quantity of water-based refrigerant gel 23 is also a safe choice for the pet or the pet owner in case the primary quantity of water-based refrigerant gel 23 is leaked due to any accident that may damage the primary hermetically-sealed pouch 22. So, the primary quantity of water-based refrigerant gel 23 is retained by the primary hermetically-sealed pouch 22 to safekeep the primary quantity of water-based refrigerant gel 23 within the back-bracing pad 1. Further, the primary quantity of water-based refrigerant gel 23 has a weight and density heavy enough to weight down the back-bracing pad 1, which enables the back-bracing pad 1 to serve as a gel-weighted blanket for the pet. In some embodiments, multiple primary gel packs may be provided within the back-bracing pad 1 to further improve the flexibility of the back-bracing pad 1.

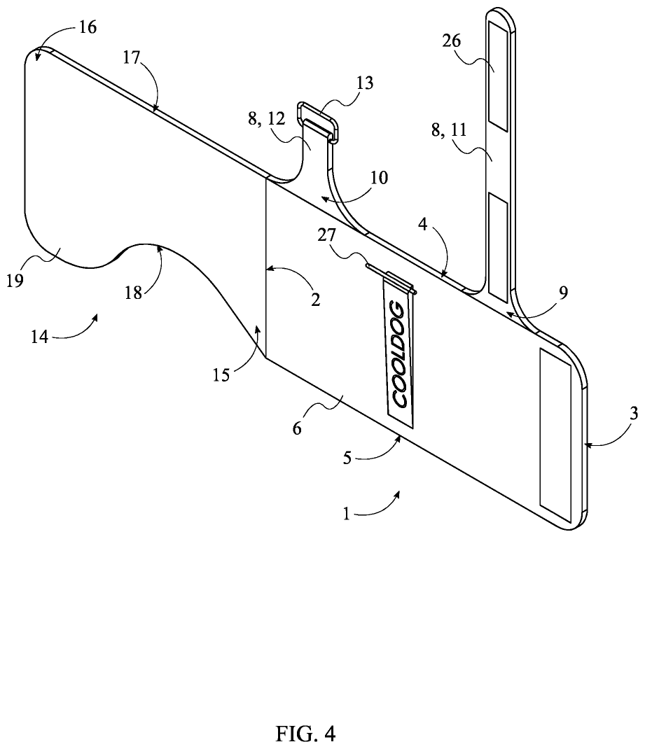

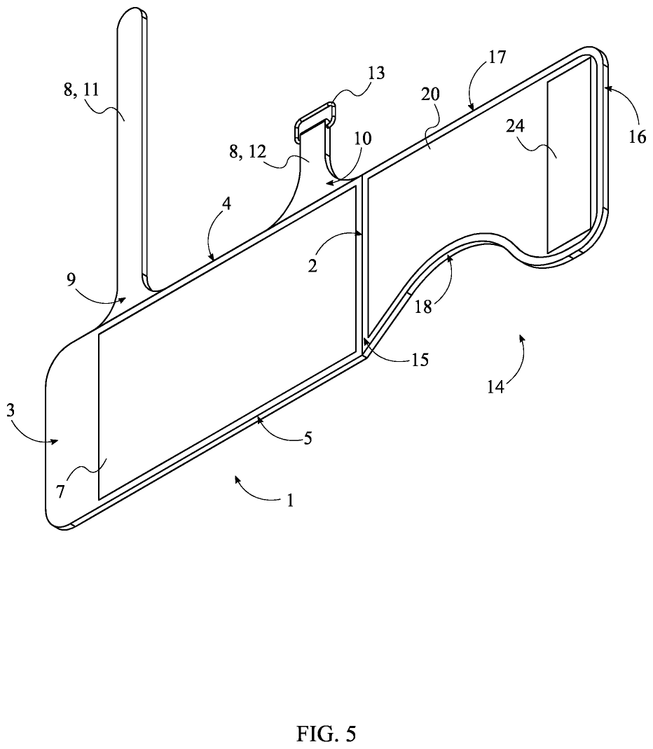

To further improve the pet's comfort when wearing the present invention, the back-bracing pad 1 is designed to make the heat transfer between the pet's body and the at least one primary gel pack 21 as comfortable as possible. So, the back-bracing pad 1 may further comprise a pad outer layer 6 and a pad inner layer 7, as can be seen in FIGS. 4, 5, and 8. The pad outer layer 6 is preferably a thermal-insulative fabric to prevent the at least one primary gel-pack from warming up too fast, thus keeping the at least one primary gel pack 21 cool for longer. The pad inner layer 7 is preferably a moisture-wicking fabric designed to soak up any moisture or sweat that may result from the heat transfer between the at least one primary gel pack 21 and the pet's body. The pad outer layer 6 is coextensive with the pad inner layer 7. In addition, the pad outer layer 6 is perimetrically connected to the pad inner layer 7. So, the pad outer layer 6 and the pad inner layer 7 form a closed pad structure. Furthermore, the at least one primary gel pack 21 is positioned in between the pad outer layer 6 and the pad inner layer 7 to keep the at least one primary gel pack 21 within the back-bracing pad 1. In other embodiments, the back-bracing pad 1 may include additional layers made from different materials that increase the comfort to the pet while wearing the present invention.

To make the present invention fit the anatomy of the pet, the girth-bracing band 14 is designed to accommodate the different anatomical features of the pet. As can be seen in FIG. 1 through 3, 4, and 5, the girth-bracing band 14 may further comprise a first lengthwise edge 17 and a second lengthwise edge 18 that preferably correspond to the long edges of the girth-bracing band 14. The first lengthwise edge 17 is preferably a straight edge, while the second lengthwise edge 18 is a concave edge. The concave edge accommodates the various anatomical features around the pet's abdomen, such as the reproductive organs of the pet. So, the first lengthwise edge 17 is positioned coincident with the cranial edge 4. On the other hand, the second lengthwise edge 18 is positioned coincident with the caudal edge 5. This enables the girth-bracing band 14 to be wrapped around the pet's abdomen without causing discomfort to the pet when wearing the present invention.

Furthermore, to enable the girth-bracing band 14 to be releasably fastened around the pet's abdomen, the present invention may further comprise a band fastener 24. As can be seen in FIG. 1 through 3, 4, and 5, the band fastener 24 is a fastener that enables the girth-bracing band 14 to be releasably fastened. The band fastener 24 can be any releasable fastener, such as a hook and loop fastener. The band fastener 24 is mounted onto the girth-bracing band 14, adjacent to the second band end 16 to enable the girth-bracing band 14 to be attached onto the back-bracing pad 1. To fasten the girth-bracing band 14 to the back-bracing pad 1, the band fastener 24 is attached onto the back-bracing pad 1, adjacent to the second lateral edge 3. This enables the girth-bracing band 14 to be wrapped under the pet's abdomen. The band fastener 24 can also enable the girth-bracing band 14 to accommodate different sizes of pets. To do so, the band fastener 24 is attached onto the back-bracing pad 1 at different locations on the back-bracing pad 1. In other embodiments, different fasteners can be utilized to enable the releasable fastening of the girth-bracing band 14 to the back-bracing pad 1.

In addition to the releasable fastening capabilities of the girth-bracing band 14, the girth-bracing band 14 can also be able to cool down the pet's body under the pet's abdomen. As can be seen in FIG. 6 through 9, the present invention may further comprise at least one secondary gel pack 25. The at least one secondary gel pack 25 may include a structure similar to the at least one primary gel pack 21 but is designed to fit inside the girth-bracing band 14 to cool down the pet's abdomen. Moreover, the at least one secondary gel pack 25 is integrated into the girth-bracing band 14. So, when the girth-bracing band 14 is wrapped under the pet's abdomen, the at least one secondary gel pack 25 is in thermal contact with the pet's abdomen. In other embodiments, like the at least one primary gel pack 21, multiple secondary gel packs can be distributed within the girth-bracing band 14 to increase the flexibility of the girth-bracing band 14.

As can be seen in FIG. 9, to maintain the at least one secondary gel pack 25 cool and to protect the pet's body, the girth-bracing band 14 may further comprise a band outer layer 19 and a band inner layer 20, similar to the back-bracing pad 1. Like the pad outer layer 6, the band outer layer 19 is preferably a thermal-insulative fabric. Also, like the pad inner layer 7, the band inner layer 20 is preferably a moisture-wicking fabric. This keeps the at least one secondary gel pack 25 cool for longer and keeps the pet's body comfortable during the heat transfer. Further, like the pad outer layer 6 and the pad inner layer 7, the band outer layer 19 is coextensive with the band inner layer 20. The band outer layer 19 is also perimetrically connected to the band inner layer 20. So, the band outer layer 19 and the band inner layer 20 form a closed band structure that encloses the at least one secondary gel pack 25. Furthermore, the at least one secondary gel pack 25 is positioned in between the band outer layer 19 and the band inner layer 20, thus cooling down the abdomen area of the pet's body when the girth-bracing band 24 is wrapped under the pet's abdomen. In other embodiments, additional gel packs can be provided in different locations within the present invention.

Like the girth-bracing band 14, the neck-bracing strap 8 can be releasably fastened to facilitate the attachment and removal of the present invention. So, the neck-bracing strap 8 may further comprise a first strap member 11, a second strap member 12, and a strap eyelet 13. As can be seen in FIG. 1 through 3, 4, and 5, the first strap member 11 is preferably the elongated member that enables the adjustment of the working length of the neck-bracing strap 8. The second strap member 12 serves to retain the strap eyelet 13 which facilitates the adjustment of the working length of the neck-bracing strap 8. The first strap member 11 is positioned coincident to the first strap end 9, while the second strap member 12 is positioned coincident to the second strap end 10. This provides enough space between the first strap member 11 and the second strap member 12 to accommodate the pet's neck. Moreover, the strap eyelet 13 is terminally connected to the second strap member 12, offset from the second strap end 10, so that the strap eyelet 13 is kept separate from the back-bracing pad 1. Further, the first strap member 11 is looped through the strap eyelet 13, offset from the first strap end 9, to enable the user to adjust the working length of the neck-bracing strap 8.

Further, to lock a desired working length of the neck-bracing strap 8, the present invention may further comprise a strap fastener 26. As can be seen in FIG. 1 through 3, 4, and 5, the strap fastener 26 further facilitates the length adjustment of the neck-bracing strap 8 to accommodate the pet's neck by locking the desired working length of the neck-bracing strap 8. The strap fastener 26 can be any fastener that can be releasably fastened, such as a hook and loop fastener. The strap fastener 26 is mounted onto the first strap member 11, offset from the first strap end 9, to enable the strap fastener 26 to be attached onto the first strap member 11 at different locations along the first strap member 11. So, to fasten the neck-bracing strap 8, the strap fastener 26 is attached onto the first strap member 11, adjacent to the first strap end 9. This also enables the pet owner to adjust the working length of the neck-bracing strap 8 by attaching the strap fastener 26 onto the first strap member 11 at different positions along the first strap member 11. In other embodiments, the strap fastener 26 can be replaced with different types of fasteners that do not require the first strap member 11 to be looped through the strap eyelet 13.

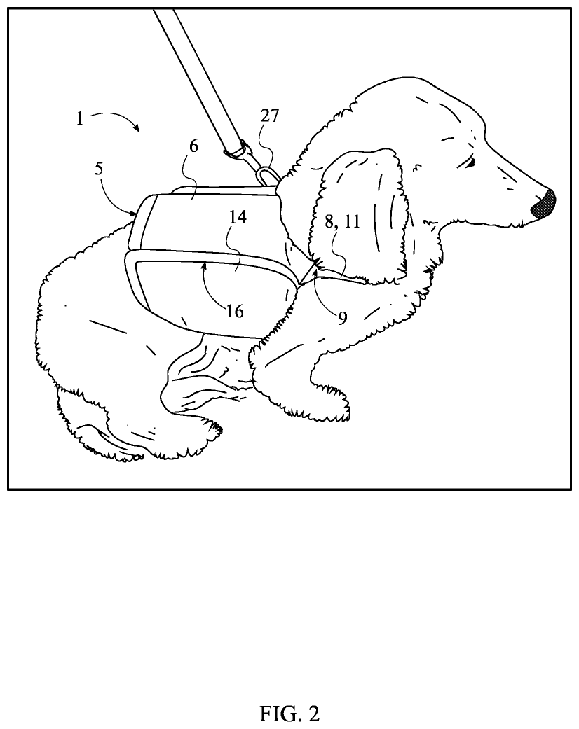

Finally, to enable the present invention to be tethered to a leash or similar control device that lets the pet owner to control the movement of the pet, the present invention may further comprise at least one leash-attachment eyelet 27. As can be seen in FIG. 2 through 4, the at least one leash-attachment eyelet 27 is designed to receive the hook of the leash to tether the present invention to the handle of the leash. The at least one leash-attachment eyelet 27 is positioned adjacent to the cranial edge 4 so that the pet is pulled from the neck using the leash. The at least one leash-attachment eyelet 27 is also centrally positioned along the cranial edge 4 to prevent the leash from twisting the present invention around the pet's body. Further, the at least one leash-attachment eyelet 27 is externally mounted onto the back-bracing pad to secure the at least one leash-attachment eyelet 27 to the back-bracing pad 1. In other embodiments, the present invention can utilize different mechanisms to enable the pet owner to attach a leash to the present invention.

In an exemplary embodiment, the pet owner places the present invention into a freezer or refrigerator prior to use. The at least one primary gel pack 21 and/or the at least one secondary gel pack 25 freeze over time, and the pet owner can retrieve the present invention from the freezer once the at least one primary gel pack 21 and/or the at least one secondary gel pack 25 are cool enough. The pet owner can warm up the present invention if the present invention is too cold by rubbing on the back-bracing pad 1 and/or the girth-bracing band 14, if necessary. Then, the pet owner places the back-bracing pad 1 onto the pet's back, aligning the cranial edge 4 with the pet's neck. Then, the pet owner can either fasten the neck-bracing strap 8 or the girth-bracing band 14. The pet owner can fasten the neck-bracing strap 8 by first looping the first strap member 11 through the strap eyelet 13, and then attaching the strap fastener 26 onto the first strap member 11 at a desired length that accommodates the pet's neck. The pet owner can also fasten the girth-bracing band 14 by first wrapping the girth-bracing band 14 under the pet's abdomen, and then attaching the band fastener 24 onto the back-bracing pad 1. Finally, the pet owner attaches a leash to the at least one leash-attachment eyelet 27 to control the pet. As can be seen in FIG. 1 through 3, the user is then ready to take the pet for a walk. The pet remains cool and calm even in hot days due to the at least one primary gel pack 21 and/or the at least one secondary gel pack 25 weighting down the present invention against the pet's body. When finished, the pet owner can easily remove the present invention from the pet's body by releasing both the strap fastener 26 and the band fastener 24. Then, the pet owner can put the present invention back into the freezer or the refrigerator for the next use.

Although the invention has been explained in relation to its preferred embodiment, it is to be understood that many other possible modifications and variations can be made without departing from the spirit and scope of the invention as hereinafter claimed.

What is claimed is:

1. A freezable pet harness comprises:
a back-bracing pad;
a neck-bracing strap;
a girth-bracing band;
at least one primary gel pack;
the back-bracing pad comprising a first lateral edge, a second lateral edge, a cranial edge, and a caudal edge;
the neck-bracing strap comprising a first strap end and a second strap end;
the girth-bracing band comprising a first band end and a second band end;
the first lateral edge and the second lateral edge being positioned opposite to each other across the back-bracing pad;
the cranial edge and the caudal edge being positioned opposite to each other across the back-bracing pad;
the cranial edge and the caudal edge being positioned in between the first lateral edge and the second lateral edge;
the at least one primary gel pack being integrated into the back-bracing pad;
the first strap end and the second strap end being connected adjacent to the cranial edge;
the first strap end and the second strap end being positioned offset from each other;
the first band end being connected adjacent to the first lateral edge; and,
the second band end being attached adjacent to the second lateral edge.

2. The freezable pet harness as claimed in claim 1 comprising:
the at least one primary gel pack comprising a primary hermetically-sealed pouch and a primary quantity of water-based refrigerant gel; and,
the primary quantity of water-based refrigerant being retained by the primary hermetically-sealed pouch.

3. The freezable pet harness as claimed in claim 1 comprising:
the back-bracing pad further comprising a pad outer layer and a pad inner layer;
the pad outer layer being a thermal-insulative fabric;
the pad inner layer being a moisture-wicking fabric;
the pad outer layer being coextensive with the pad inner layer; and,
the pad outer layer being perimetrically connected to the pad inner layer.

4. The freezable pet harness as claimed in claim 3 comprising:
the at least one primary gel pack being positioned in between the pad outer layer and the pad inner layer.

5. The freezable pet harness as claimed in claim 1 comprising:
the girth-bracing band further comprising a first lengthwise edge and a second lengthwise edge;
the first lengthwise edge being a straight edge;
the second lengthwise edge being a concave edge;
the first lengthwise edge being positioned coincident with the cranial edge; and,
the second lengthwise edge being positioned coincident with the caudal edge.

6. The freezable pet harness as claimed in claim 1 comprising:
a band fastener;
the band fastener being mounted onto the girth-bracing band, adjacent to the second band end; and,
the band fastener being attached onto the back-bracing pad, adjacent to the second lateral edge.

7. The freezable pet harness as claimed in claim 1 comprising:
at least one secondary gel pack; and,
the at least one secondary gel pack being integrated into the girth-bracing band.

8. The freezable pet harness as claimed in claim 7 comprising:
the girth-bracing band further comprising a band outer layer and a band inner layer;
the band outer layer being a thermal-insulative fabric;
the band inner layer being a moisture-wicking fabric;
the band outer layer being coextensive with the band inner layer; and,
the band outer layer being perimetrically connected to the band inner layer.

9. The freezable pet harness as claimed in claim 8 comprising:
the at least one secondary gel pack being positioned in between the band outer layer and the band inner layer.

10. The freezable pet harness as claimed in claim 1 comprising:
the neck-bracing strap further comprising a first strap member, a second strap member, and a strap eyelet;
the first strap member being positioned coincident to the first strap end;
the second strap member being positioned coincident to the second strap end;
the strap eyelet being terminally connected to the second strap member, offset from the second strap end; and,
the first strap member being looped through the strap eyelet, offset from the first strap end.

11. The freezable pet harness as claimed in claim 10 comprising:
a strap fastener;
the strap fastener being mounted onto the first strap member, offset from the first strap end; and,
the strap fastener being attached onto the first strap member, adjacent to the first strap end.

12. The freezable pet harness as claimed in claim 1 comprising:
at least one leash-attachment eyelet;
the at least one leash-attachment eyelet being positioned adjacent to the cranial edge;
the at least one leash-attachment eyelet being centrally positioned along the cranial edge; and,
the at least one leash-attachment eyelet being externally mounted onto the back-bracing pad.

13. A freezable pet harness comprises:
a back-bracing pad;
a neck-bracing strap;
a girth-bracing band;
at least one primary gel pack;
the back-bracing pad comprising a first lateral edge, a second lateral edge, a cranial edge, and a caudal edge;
the neck-bracing strap comprising a first strap end and a second strap end;
the girth-bracing band comprising a first band end and a second band end;
the at least one primary gel pack comprising a primary hermetically-sealed pouch and a primary quantity of water-based refrigerant gel;
the first lateral edge and the second lateral edge being positioned opposite to each other across the back-bracing pad;
the cranial edge and the caudal edge being positioned opposite to each other across the back-bracing pad;
the cranial edge and the caudal edge being positioned in between the first lateral edge and the second lateral edge;
the at least one primary gel pack being integrated into the back-bracing pad;
the first strap end and the second strap end being connected adjacent to the cranial edge;
the first strap end and the second strap end being positioned offset from each other;
the first band end being connected adjacent to the first lateral edge;
the second band end being attached adjacent to the second lateral edge; and,
the primary quantity of water-based refrigerant being retained by the primary hermetically-sealed pouch.

14. The freezable pet harness as claimed in claim 13 comprising:
the back-bracing pad further comprising a pad outer layer and a pad inner layer;
the pad outer layer being a thermal-insulative fabric;
the pad inner layer being a moisture-wicking fabric;
the pad outer layer being coextensive with the pad inner layer;
the pad outer layer being perimetrically connected to the pad inner layer; and,
the at least one primary gel pack being positioned in between the pad outer layer and the pad inner layer.

15. The freezable pet harness as claimed in claim 13 comprising:
the girth-bracing band further comprising a first lengthwise edge and a second lengthwise edge;
the first lengthwise edge being a straight edge;
the second lengthwise edge being a concave edge;
the first lengthwise edge being positioned coincident with the cranial edge; and,
the second lengthwise edge being positioned coincident with the caudal edge.

16. The freezable pet harness as claimed in claim 13 comprising:
a band fastener;
the band fastener being mounted onto the girth-bracing band, adjacent to the second band end; and,
the band fastener being attached onto the back-bracing pad, adjacent to the second lateral edge.

17. The freezable pet harness as claimed in claim 13 comprising:
at least one secondary gel pack;
the girth-bracing band further comprising a band outer layer and a band inner layer;
the at least one secondary gel pack being integrated into the girth-bracing band;
the band outer layer being a thermal-insulative fabric;
the band inner layer being a moisture-wicking fabric;
the band outer layer being coextensive with the band inner layer; and,
the band outer layer being perimetrically connected to the band inner layer.

18. The freezable pet harness as claimed in claim 17 comprising:
the at least one secondary gel pack being positioned in between the band outer layer and the band inner layer.

19. The freezable pet harness as claimed in claim 13 comprising:
a strap fastener;
the neck-bracing strap further comprising a first strap member, a second strap member, and a strap eyelet;
the first strap member being positioned coincident to the first strap end;
the second strap member being positioned coincident to the second strap end;
the strap eyelet being terminally connected to the second strap member, offset from the second strap end;
the first strap member being looped through the strap eyelet, offset from the first strap end;
the strap fastener being mounted onto the first strap member, offset from the first strap end; and,
the strap fastener being attached onto the first strap member, adjacent to the first strap end.

20. The freezable pet harness as claimed in claim 13 comprising:
at least one leash-attachment eyelet;
the at least one leash-attachment eyelet being positioned adjacent to the cranial edge;
the at least one leash-attachment eyelet being centrally positioned along the cranial edge; and,
the at least one leash-attachment eyelet being externally mounted onto the back-bracing pad.
